(19) United States
(12) Patent Application Publication
(10) Pub. No.: US 2022/0280037 A1
(43) Pub. Date: Sep. 8, 2022

(54) OPHTHALMIC IMAGING USING A HEAD-WORN DEVICE

(71) Applicant: Snap Inc., Santa Monica, CA (US)

(72) Inventors: Daniel Colascione, Seattle, WA (US); Kenneth Kubala, Boulder, CO (US); Brent Mills, Vashon, WA (US)

(21) Appl. No.: 17/651,857

(22) Filed: Feb. 21, 2022

Related U.S. Application Data
(60) Provisional application No. 63/156,184, filed on Mar. 3, 2021.

(51) Int. Cl. A61B 3/14; A61B 3/00

(52) U.S. Cl. CPC A61B 3/14; A61B 3/005; A61B 3/0033; A61B 3/0025

(57) ABSTRACT

Disclosed is a method of conducting an ophthalmic evaluation using an augmented reality or virtual reality head-worn device including one or more display devices and one or more cameras facing towards an eye of the user in use. The method comprises displaying an ophthalmic evaluation user interface item on a display device, receiving user selection of the user interface item, illuminating an eye of the user, capturing at least one image of the eye of the user; and analyzing the at least one image to assess the health of the eye of the user.

### RELATED APPLICATION DATA

This application claims the benefit of priority to U.S. Provisional Patent Application Ser. No. 63/156,184 filed on Mar. 3, 2021, the contents of which are incorporated herein as if explicitly set forth.

### TECHNICAL FIELD

The present disclosure relates generally to wearable devices and to vision assessments performed using wearable devices.

### BACKGROUND

A head-worn device may be implemented with a transparent or semi-transparent display through which a user of the wearable device can view the surrounding environment, Such devices enable a user to see through the transparent or semi-transparent display to view the surrounding environment, and to also see objects (e.g., virtual objects such as 3D renderings, images, video, text, and so forth) that are generated for display to appear as a part of, and/or overlaid upon, the surrounding environment. This is typically referred to as “augmented reality.”

A head-worn device may additionally completely occlude a user's visual field and display a virtual environment through which a user may move or be moved. This is typically referred to as “virtual reality.” As used herein, the term “augmented reality” or “AR” refers to both augmented reality and virtual reality as traditionally understood, unless the context indicates otherwise.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a perspective view of a wearable device, in accordance with some examples.

FIG. 2 is a block diagram illustrating a networked system including details of a camera device, in accordance with one example.

FIG. 3 illustrates a wearable device including a waveguide corresponding to an integrated display, in accordance with one example.

FIG. 4 is a flowchart illustrating a process for conducting an ophthalmic assessment, in accordance with some examples.

FIG. 5 is block diagram showing a software architecture within which the present disclosure may be implemented, in accordance with some examples.

FIG. 6 is a diagrammatic representation of a machine, in the form of a computer system within which a set of instructions may be executed for causing the machine to perform any one or more of the methodologies discussed, in accordance with some examples.

### DETAILED DESCRIPTION

A wearable device implemented with a transparent or semi-transparent display enables a user to see through the transparent or semi-transparent display to view the surrounding environment. In addition, the wearable device may enable the user to see objects (e.g., virtual objects such as 3D renderings, images, video, text, and so forth) that are generated for display to appear as a part of, and/or overlaid upon, the surrounding environment. Such a wearable device may provide an augmented reality experience for the user.

The wearable device includes an integrated display for displaying objects (e.g., virtual objects) to the wearer of the device. The integrated display may for example include a waveguide that receives a light beam from a projector but any appropriate display for presenting augmented or virtual content to the wearer may be used.

The wearable device in use displays a virtual eye chart to the wearer, typically but not necessarily in conjunction with an associated portable computing device. The eye chart is rendered at the appropriate apparent distance and at the appropriate apparent size to present a standard eye chart, which can be used to assess the user's vision using familiar techniques, but without requiring a visit to the optometrist. Audio prompts may be provided to step the user through the eye chart, and the user's responses can be captured and recognized by the wearable device or the portable device using voice recognition techniques, hand gestures or through some other input device associated with the wearable device, such as a smartphone or other portable computing device. The responses by the user can be compared against expected responses to the prompts and the user's performance can be scored. An overall visual score can then be generated for each eye, which can be presented to the user or transmitted to a medical office.

In addition, the wearable device may comprise one or more cameras facing towards the wearer's eyes in use. These cameras may be used to image the wearer's eyes to determine if there may be any ocular conditions requiring further attention. Additionally, the wearable device may comprise one or more light emitters in the visual or near visual spectrum that may be used to illuminate an exterior or interior surface of the eve to assist with this determination. Furthermore, the light emitters may be configured to project a pattern onto the wearer's retinas, which can then be captured and analyzed by the cameras facing towards the wearer's eyes.

In some examples, provided is a method of conducting an ophthalmic evaluation using a head-worn device including one or more display devices and one or more cameras facing towards an eye of a user in use. The method includes displaying an ophthalmic evaluation user interface item, receiving user selection of the user interface item, illuminating an eye of the user, capturing at least one image of the eye of the user, analyzing the at least one image to assess the health of the eye of the user, and providing an output of the analysis of the at least one image to the user. The illumination may be provided by light emitted from the one or more display devices. The head-worn device may also light emitters, the illumination being provided by the light emitters.

The method may further include generating audible ophthalmic evaluation prompts on an audio speaker associated with the head-worn device, and determining compliance with the audible ophthalmic evaluation prompts prior to capturing the at least one image. The audible ophthalmic evaluation prompts may comprise instructions to look in a certain direction and determining compliance may be performed by image recognition performed on frames of an image stream received from the one or more cameras. Determining compliance may also be performed by receipt of confirmatory user input.

The method may also include further includes displaying a feature on the display device, where the ophthalmic evaluation prompts comprise instructions to look at the feature displayed on the display device.

In some examples, capturing of at least one image of the eye of the user comprises capturing multiple images of the eye of the user, and generating a composite image of the eye of the user from the multiple images. The method may also include generating audible ophthalmic evaluation prompts on an audio speaker associated with the head-worn device, and determining compliance with each audible ophthalmic evaluation prompt prior to capturing each of the multiple images.

The ophthalmic evaluation user interface item may include a plurality of ophthalmic evaluations available for user selection, the method further includes receiving user selection of a specific ophthalmic evaluation, and setting illumination and camera exposure settings based on the specific ophthalmic evaluation.

In some examples, provided is a computer system including a head-worn device, one or more processors, one or more display devices, and one or more cameras facing towards an eye of a user in use. The computer system also includes a memory storing instructions that, when executed by the one or more processors, configure the computer system to perform operations corresponding to the methods disclosed above, including but not limited to displaying an ophthalmic evaluation user interface item on a display device, receiving user selection of the user interface item, illuminating an eye of the user, capturing at least one image of the eye of the user, and analyzing the at least one image to assess the health of the eye of the user.

In some examples, provided is a non-transitory computer-readable storage medium, the computer-readable storage medium including instructions that when executed by a computer system including a head-worn device including one or more display devices and one or more cameras facing towards an eye of a user in use, cause the computer system to perform operations corresponding to the methods disclosed above, including but not limited to displaying an ophthalmic evaluation user interface item on a display device, receiving user selection of the user interface item, illuminating an eye of the user, capturing at least one image of the eye of the user, and analyzing the at least one image to assess the health of the eye of the user.

Other technical features may be readily apparent to one skilled in the art from the following figures, descriptions, and claims.

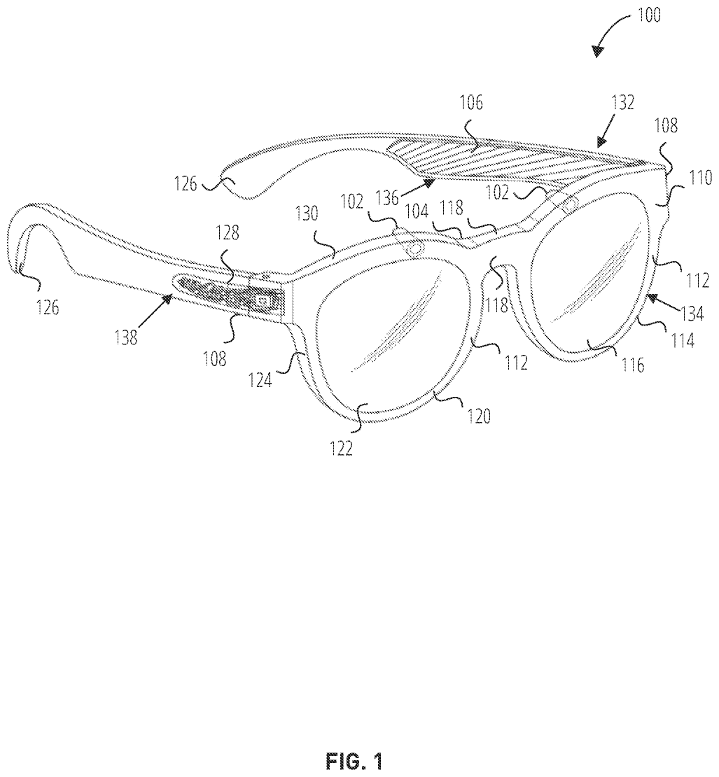

FIG. 1 is perspective view of a wearable device (e.g., glasses 100), in accordance with some examples. The glasses 100 can include a frame 132 made from any suitable material such as plastic or metal, including any suitable shape memory alloy. In one or more examples, the frame 132 includes a front piece 134 including a first or left optical element holder 114 (e.g., a display or lens holder) and a second or right optical element holder 120 connected by a bridge 118. The front piece 134 additionally includes a left end portion 110 and a right end portion 124. A first or left optical element 116 and a second or right optical element 122 can be provided within respective left optical element holder 114 and right optical element holder 120. Each of the right optical element 122 and the left optical element 116 can be a lens, a display, a display assembly, or a combination of the foregoing. Any suitable display assembly can be provided in the glasses 100.

The frame 132 additionally includes a left arm or temple piece 136 and a right arm or temple piece 138 coupled to the respective left end portion 110 and the right end portion 124 of the front piece 134 by any suitable means such as a hinge (not shown), so as to be coupled to the front piece 134, or rigidly or fixably secured to the front piece 134 so as to be integral with the front piece 134. In one or more implementations, each of the temple piece 136 and the temple piece 138 includes a first portion 108 that is coupled to the respective left end portion 110 or right end portion 124 of the front piece 134 and any suitable second portion 126 for coupling to the ear of the user. In one example, the front piece 134 can be formed from a single piece of material, so as to have a unitary or integral construction. In one example, such as illustrated in FIG. 1, the entire frame 132 can be formed from a single piece of material so as to have a unitary or integral construction.

The glasses 100 can include a computing device, such as a computer 128, which can be of any suitable type so as to be carried by the frame 132 and, in one or more examples, of a suitable size and shape, so as to be at least partially disposed in one of the temple piece 136 and the temple piece 138. In one or more examples, as illustrated in FIG. 1, the computer 128 is sized and shaped similar to the size and shape of one of the temple piece 138 (e.g., or the temple piece 136), and is thus disposed almost entirely if not entirely within the structure and confines of such temple piece 138. In one or more examples, the computer 128 is disposed in both of the temple piece 136 and the temple piece 138. The computer 128 can include one or more processors with memory, wireless communication circuitry, and a power source. As discussed below, the computer 128 comprises low-power circuitry, high-speed circuitry, and a display processor. Various other examples may include these elements in different configurations or integrated together in different ways. Additional details of aspects of computer 128 may be implemented as illustrated by the data processor 202 discussed below.

The computer 128 additionally includes a battery 106 or other suitable portable power supply. In one example, the battery 106 is disposed in one of the temple piece 136 or the temple piece 138. In the glasses 100 shown in FIG. 1, the battery 106 is shown as being disposed in left temple piece 136 and electrically coupled using the connection 130 to the remainder of the computer 128 disposed in the right temple piece 138. The glasses 100 can include a connector or port (not shown) suitable for charging the battery 106 accessible from the outside of frame 132, a wireless receiver, transmitter or transceiver (not shown), or a combination of such devices.

In one or more implementations, the glasses 100 include cameras 102. Although two cameras are depicted, other examples contemplate the use of a single or additional (i.e., more than two) cameras. In one or more examples, the glasses 100 include any number of input sensors or peripheral devices in addition to the cameras 102. The front piece 134 is provided with an outward facing, forward-facing or front or outer surface 112 that faces forward or away from the user when the glasses 100 are mounted on the face of the user, and an opposite inward-facing, rearward-facing or rear or inner surface 104 that faces the face of the user when the glasses 100 are mounted on the face of the user. Such sensors can include inwardly-facing video sensors or digital imaging modules such as cameras be mounted on or provided within the inner surface 104 of the front piece 134 or elsewhere on the frame 132 so as to be facing the user, and outwardly-facing video sensors or digital imaging modules such as the cameras 102 that can be mounted on or provided with the outer surface 112 of the front piece 134 or elsewhere on the frame 132 so as to be facing away from the user. Such sensors, peripheral devices or peripherals can additionally include biometric sensors, location sensors, or any other such sensors.

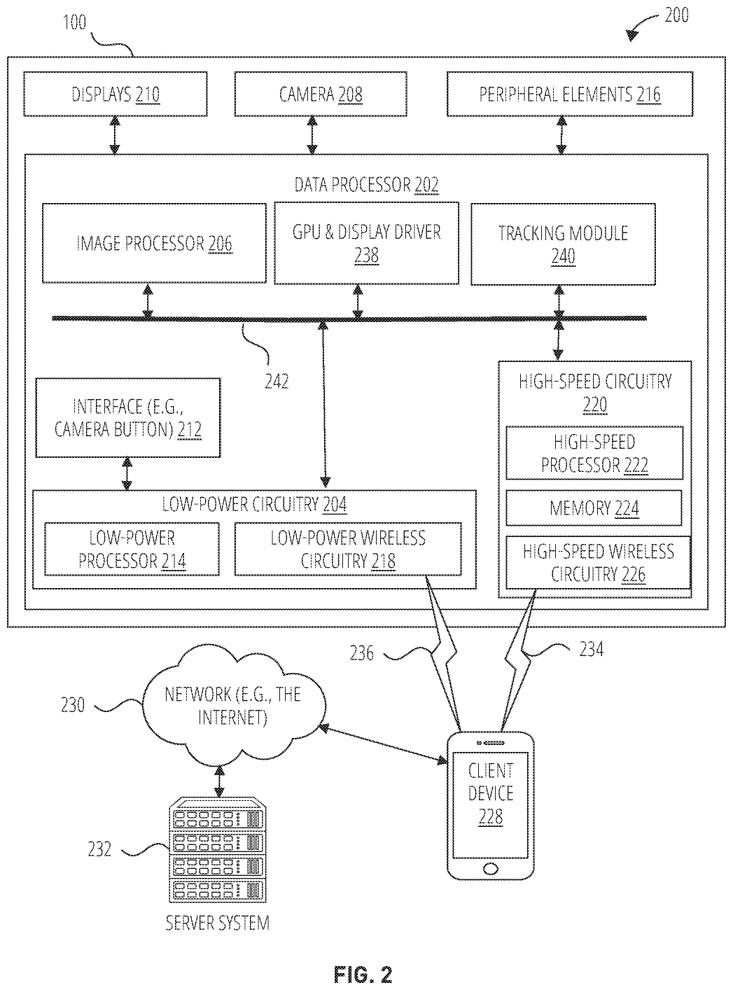

FIG. 2 is a block diagram illustrating a networked system 200 including details of the glasses 100, in accordance with some examples.

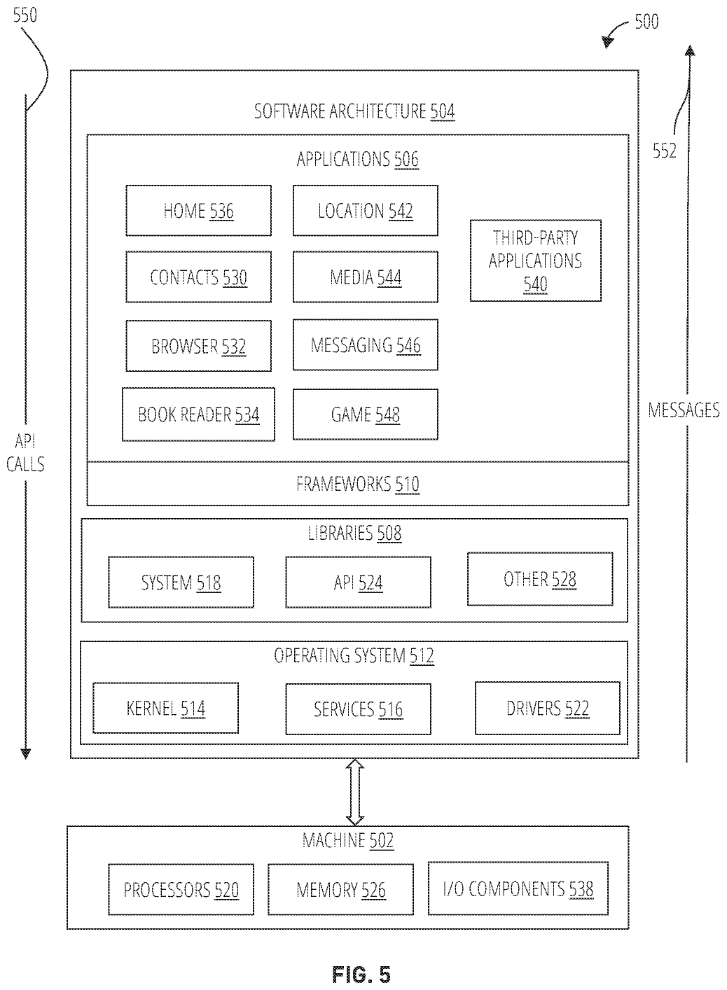

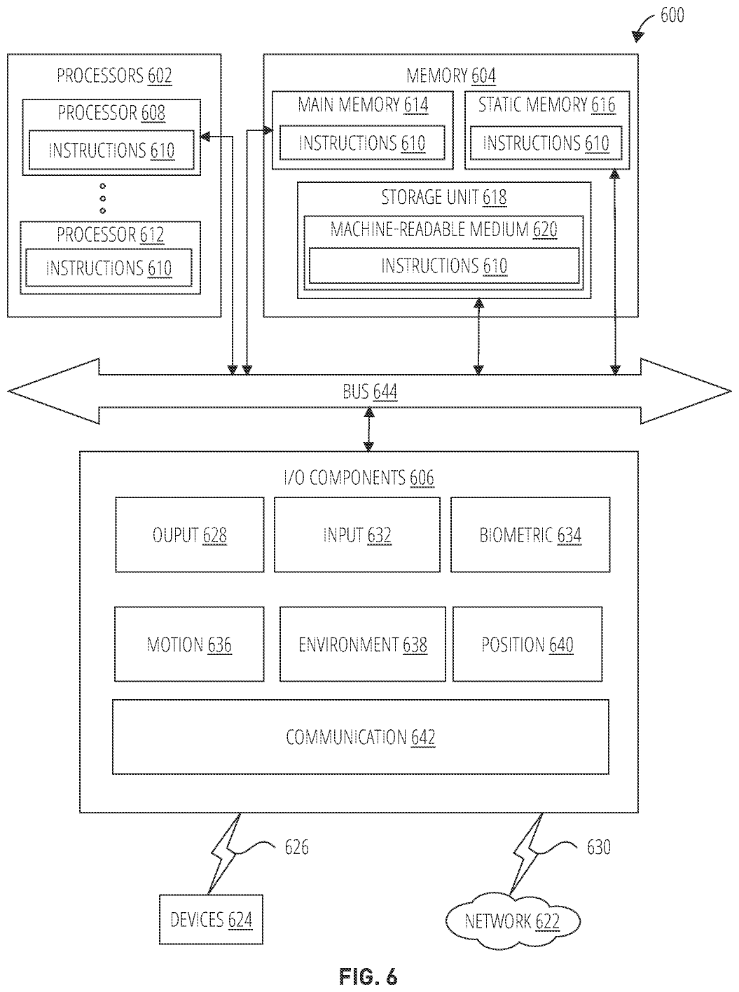

The networked system 200 includes the glasses 100, a client device 228, and a server system 232. The client device 228 may be a smartphone, tablet, phablet, laptop computer, access point, or any other such device capable of connecting with the glasses 100 using both a low-power wireless connection 236 and a high-speed wireless connection 234. The client device 228 is connected to the server system 232 via the network 230. The network 230 may include any combination of wired and wireless connections. The server system 232 may be one or more computing devices as part of a service or network computing system. The client device 228 and any elements of the server system 232 and network 230 may be implemented using details of the software architecture 504 or the machine 600 described in FIG. 5 and FIG. 6.

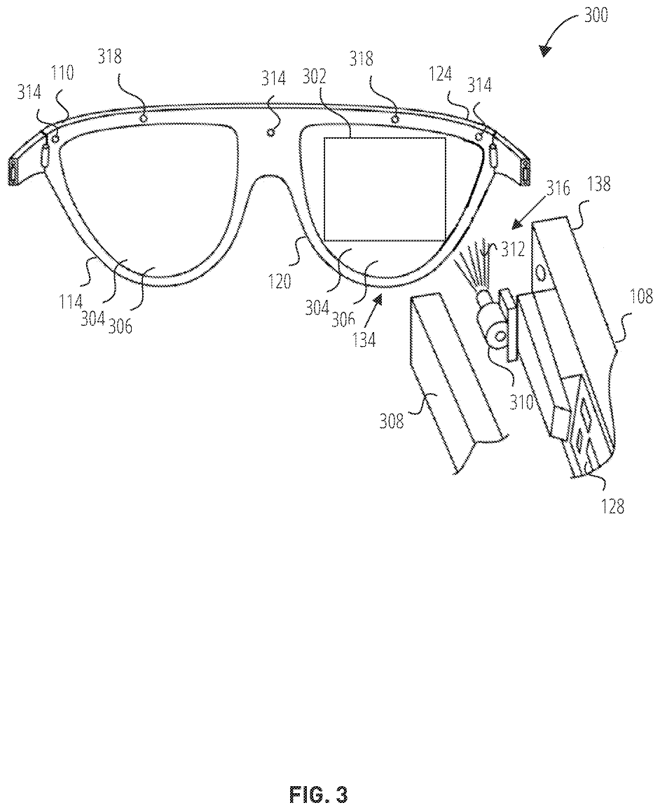

The glasses 100 include a data processor 202, displays 210, one or more cameras 208, additional peripheral elements 216. The peripheral elements 216 may include microphones, audio speakers, biometric sensors, additional sensors, or additional display elements integrated with the data processor 202. Examples of the peripheral elements 216 are discussed further with respect to FIG. 5 and FIG. 6. For example, the peripheral elements 216 may include any I/O components 606 including output components 628, motion components 636, or any other such elements described herein. Examples of the displays 210 is discussed in FIG. 3. In the particular examples described herein, the displays 210 include a display for each one of a user's left and right eyes.

The data processor 202 includes an image processor 206 (e.g., a video processor), a GPU & display driver 238, a tracking module 240, an interface 212, low-power circuitry 204, and high-speed circuitry 220. The components of the data processor 202 are interconnected by a bus 242.

The interface 212 refers to any source of a user command that is provided to the data processor 202. in one or more examples, the interface 212 is a physical button on a camera that, when depressed, sends a user input signal from the interface 212. to a low-power processor 214. A depression of such a camera button followed by an immediate release may be processed by the low-power processor 214 as a request to capture a single image. A depression of such a camera button for a first period of time may be processed by the low-power processor 214 as a request to capture video data while the button is depressed, and to cease video capture when the button is released, with the video captured while the button was depressed stored as a single video file. In other examples, the interface 212 may be any mechanical switch or physical interface capable of accepting user inputs associated with a request for data from the camera 208, In other examples, the interface 212 may have a software component, or may be associated with a command received wirelessly from another source, such as from the client device 228.

The image processor 206 includes circuitry to receive signals from the camera 208 and process those signals from the camera 208 into a format suitable for storage in the memory 224 or for transmission to the client device 228. In one or more examples, the image processor 206 (e.g., video processor) comprises a microprocessor integrated circuit (IC) customized for processing sensor data from the camera 208, along with volatile memory used by the microprocessor in operation.

The low-power circuitry 204 includes the low-power processor 214 and the low-power wireless circuitry 218. These elements of the low-power circuitry 204 may be implemented as separate elements or may be implemented on a single IC as part of a system on a single chip, The low-power processor 214 includes logic for managing the other elements of the glasses 100. As described above, for example, the low-power processor 214 may accept user input signals from the interface 212. The low-power processor 214 may also be configured to receive input signals or instruction communications from the client device 228 via the low-power wireless connection 236. The low-power wireless circuitry 218 includes circuit elements for implementing a low-power wireless communication system. Bluetooth™ Smart, also known as Bluetooth™ low energy, is one standard implementation of a low power wireless communication system that may be used to implement the low-power wireless circuitry 218. In other examples, other low power communication systems may be used.

The high-speed circuitry 220 includes a high-speed processor 222, a memory 224, and a high-speed wireless circuitry 226. The high-speed processor 222 may be any processor capable of managing high-speed communications and operation of any general computing system needed for the data processor 202. The high-speed processor 222 includes processing resources needed for managing high-speed data transfers on the high-speed wireless connection 234 using the high-speed wireless circuitry 226. In certain examples, the high-speed processor 222 executes an operating system such as a LINUX operating system or other such operating system such as the operating system 512 of FIG. 5. In addition to any other responsibilities, the high-speed processor 222 executing a software architecture for the data processor 202 is used to manage data transfers with the high-speed wireless circuitry 226. In certain examples, the high-speed wireless circuitry 226 is configured to implement Institute of Electrical and. Electronic Engineers (IEEE) 802.11 communication standards, also referred to herein as Wi-Fi. In other examples, other high-speed communications standards may be implemented by the high-speed wireless circuitry 226.

The memory 224 includes any storage device capable of storing camera data generated by the camera 208 and the image processor 206. While the memory 224 is shown as integrated with the high-speed circuitry 220, in other examples, the memory 224 may be an independent standalone element of the data processor 202. In certain such examples, electrical routing lines may provide a connection through a chip that includes the high-speed processor 222 from image processor 206 or the low-power processor 214 to the memory 224. In other examples, the high-speed processor 222 may manage addressing of the memory 224 such that the low-power processor 214 will boot the high-speed processor 222 any time that a read or write operation involving the memory 224 is needed.

The tracking module 240 estimates a pose of the glasses 100. For example, the tracking module 240 uses image data and corresponding inertial data from the camera 208 and the position components 640, as well as (IPS data, to track a location and pose of the glasses 100 relative to a frame of reference (e.g., real-world environment). In one example, the tracking module 240 uses the sensor data to determine the three-dimensional pose of the glasses 100. The three-dimensional pose is a determined orientation and position of the glasses 100 in relation to the user's real-world environment. For example, the tracking module 240 may use images of the user's real-world environment, as well as other sensor data, to identify a relative position and orientation of the glasses 100 relative to physical objects in the real-world environment surrounding the glasses 100, using techniques such as Structure from Motion or by using an existing 3D point cloud. The tracking module 240 continually gathers and uses updated image and sensor data describing movements of the glasses 100 to determine updated three-dimensional poses of the glasses 100 that indicate changes in the relative position and. orientation relative to physical objects in the real-world environment. The tracking module 240 permits visual placement of virtual objects relative to physical objects by the glasses 100 within the field of view of the user via the displays 210.

The GPU & display driver 238 uses the three-dimensional pose of the glasses 100 to generate frames of virtual content to be presented on the displays 210. For example, the GPU & display driver 238 uses the three-dimensional pose to render a frame of virtual content such that the virtual content is presented at an appropriate orientation, position and depth in the displays 210, thereby to properly integrate the virtual content with the user's view of physical objects. The GPU appropriately scales and locates the virtual content so that the perceived size of the virtual content is consistent with the intended distance of the virtual content from the user. The GPU & display driver 238 generates updated frames of virtual content based on updated three-dimensional poses of the glasses 100, which reflect changes in the position and orientation of the user in relation to physical objects in the user's real-world environment.

Virtual objects displayed in augmented or virtual reality environments are typically displayed at a fixed focal distance, typically infinity. That is, the light rays entering the eye from any particular virtual object are parallel (for an infinite focal distance), irrespective of the perceived or virtual distance of the object based on cues such as the size of the object, its placement with respect to other objects, and stereoscopic differences between the images presented to the left and right eyes. However, conventional eye tests are typically located at a distance of 20 ft (6 m). In some cases, the glasses 100 may be provided with Alvarez lenses or with an LCD coating on the waveguide (see below) to change the focal distance to the correct focal distance from whatever standard or default focal distance is provided in the head-worn device. In other cases, the existing focal distance provided by the glasses 100 or other head-worn device might provide sufficiently accurate eye test results even if the focal distance provided by the head worn device does not correspond to the focal distance of a particular eye test.

One or more functions or operations described herein may also be performed in an application resident on the glasses 100 or on the client device 228, or on a remote server. For example, one or more functions or operations described herein may be performed by one of the applications 506 such as messaging application 546 or a custom eye test application.

FIG. 3 illustrates a wearable device (e.g., glasses 300) including a waveguide 302 forming an integrated display, in accordance with some examples. The glasses 300 can be of any suitable type, including the glasses 100, and like reference numerals have been used to describe like components of glasses 300 and glasses 100. The glasses 300 include optical lenses 304 secured within each of the left. optical element holder 114 and the right optical element holder 120. Each of the optical lenses 304 has a respective front surface (not shown) and an opposite rear surface 306.

The glasses 300 include an optical assembly 316 for displaying images to a user. In the example of FIG. 3, one optical assembly 316 is shown, but optical assemblies are normally provided for both eyes of a user (e.g., for both of the temple piece 136 and the temple piece 138). The optical assembly 316 includes an optical source such as a projector 310 that is disposed in one or both of the arms or temples of the glasses (e.g., the right temple piece 136 and left temple piece 138 of the glasses 300). The projector 310 emits light 312 toward the optical lens 304 and is enclosed by a cover 308.

The optical assembly 316 further includes a waveguide: 302. The waveguide 302 includes diffractive structures (e.g., gratings and/or optical elements). Light 312. emitted by the projector 310 encounters the diffractive structures of the waveguide 302, to provide an image on the rear surface 306 of the optical lens 304 that overlays the view of the real world seen by the user.

The glasses 300 further include inwardly-facing cameras 314, which are directed at the user's eye. Also provided are one or more light emitters that are similarly directed towards and can illuminate a user's pupil and retina. In some examples, the light emitters 318 are LED light emitters that emit an optical spectrum that is appropriate for illuminating the eye for capture of an image of the retina or pupil by the cameras 314 that can be used to identify problems or potential problems with the eye, for example glaucoma, age-related macular degeneration, or the presence of cataracts in the lens of the user's eye. In other examples, the light emitters 318 may include a grid that projects a corresponding grid onto the user's retina. An image of the grid as projected onto the retina can then be captured by one or more of the cameras 314, which can be used to evaluate the user's visual acuity by comparison with a reference or expected appearance of the grid. In yet further examples, the light emitters 318 may be tiny projectors, which can either project a spectrum of light or an image, for example, a grid, onto the user's retina. As will be appreciated each of the cameras 314 and light emitters 318 are separately addressable to provide functionality that will vary depending on which eye is under test and the nature of the test.

In another implementation, the projector 310 can be used to project a light field onto waveguide 302, which can provide sufficient illumination to permit the capture of images of the pupil or lens or retina of the pupil's eye by the cameras 314, which can be used to identify problems or potential problems with the eye, for example age-related macular degeneration, glaucoma or the presence of cataracts in the lens of the user's eye. The light field projected by the projectors 310 (one for each eye) may also for example include one or more features, such as a grid, the projection of which onto the user's retina can be used to evaluate the user's visual acuity by comparison with a reference or expected appearance of the grid.

While the cameras 314 and light emitters 318 are shown along an upper edge of the frame of the glasses, it will be appreciated that cameras 314 and light emitters 318 may be positioned elsewhere on the frame 132, for example around the left optical element holder 114 and right optical element holder 120. One or more of the cameras 314 on one or the other of the left optical element holder 114 or right optical element holder 120 may capture simultaneous or near simultaneous images, from which a composite image of one or more features of a user's eye may be constructed.

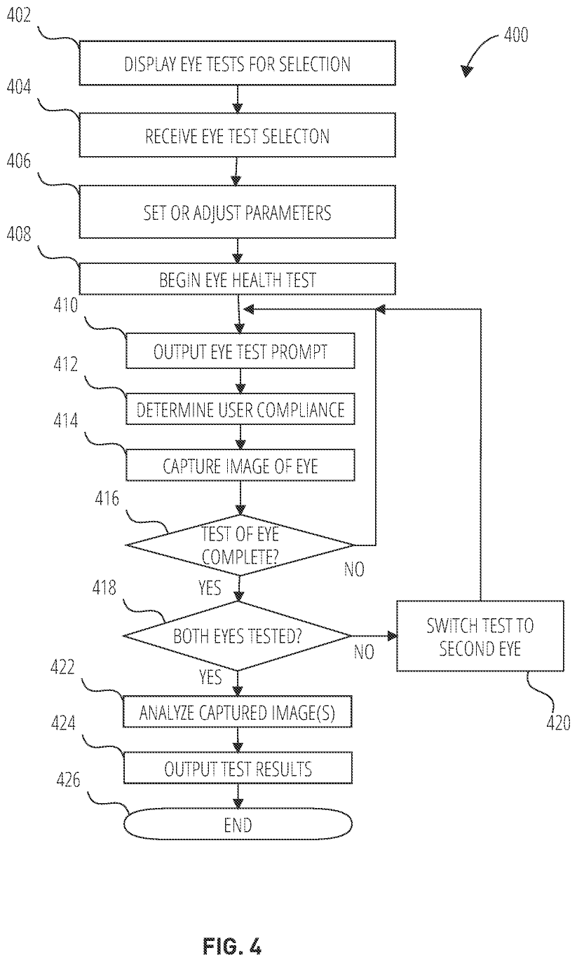

FIG. 4 is a flowchart 400 illustrating a process for conducting an ophthalmic assessment, in accordance with some examples. For explanatory purposes, the operations of the flowchart 400 are described herein as occurring in serial, or linearly. However, multiple operations of the flowchart 400 may occur in parallel. In addition, the operations of the flowchart 400 need not be performed in the order shown and/or one or more blocks of the flowchart 400 need not be performed and/or can be replaced by other operations.

Operations illustrated in FIG. 4 will typically execute on client device 228 in an application such as messaging application 546 or a dedicated ophthalmic test application. In one example, the operations are performed jointly between messaging application 546 running on the client device 228 and the data processor 202 and associated hardware in or associated with the glasses 100, 300. For the purposes of clarity, flowchart 400 is discussed herein with reference to such an example. Various implementations are of course possible, with some of the operations taking place in server system 232, or with one application calling another application or SDK for required functionality.

The method commences with the messaging application 546 displaying an ophthalmic test user interface on one or both of the displays 210 or on the display of the client device 228 at operation 402. The user interface provides one or more options for selecting ophthalmic tests to be performed using the glasses 100, 300 For example, the ophthalmic tests may include a cataract assessment, an age-related macular degeneration assessment, a glaucoma assessment, and so forth.

In one example, the user interface may provide a menu or other user interface display of visual or ophthalmic tests from which a user may choose, including for example the visual acuity tests described in US Provisional Patent Application entitled “Augmented Reality Eye Chart” filed concurrently herewith, Attorney Docket No. 4218.C35PRV, the contents of which are incorporated herein by reference as if explicitly set forth.

Selection of an ophthalmic test is then received by the messaging application 546 at operation 404. This can be done using any selection technique, including by receiving a touch input on client device 228, gesture recognition, voice input, or the like.

Upon selection of a particular ophthalmic test, the messaging application 546 sets or adjusts various parameters or modes for conducting the particular ophthalmic test at operation 406, depending on the particular requirements of the selected ophthalmic test. For example, different ophthalmic tests may require different camera exposures, focus settings or modes, settings for the amount and nature of the illumination provided by a light emitter 318 or the optical assembly 316, or for any pattern or other image to be displayed using the optical assembly 316, for example a grid to be displayed, or a display of one or more targets for the user to look at during the ophthalmic test. Additionally, different ophthalmic tests may require different steps.

The selected ophthalmic test commences at operation 408. In some instances the ophthalmic test may be completed in one perceived step (to the user) by providing appropriate illumination of or display to the eye under test followed or accompanied by capture of one or more images of the eye by one or more of the inward-facing cameras 314. In such a case, the method will proceed directly to operation 418 after the image(s) have been captured. It may in fact be advantageous to provide rapid illumination and image capture to prevent the pupil constricting in response to the illumination. Additionally, there may be preparatory steps that occur when the ophthalmic test commences, for example obscuring the user's vision by darkening the displays 210 to allow pupils to dilate, or instructing the user to go to a dark room and providing a countdown to the test.

During the test, the display for the eye that is not under test may display nothing (i.e. provide an unobstructed view) or may display a solid color (e.g. black) or other effect to obscure the other eye. In the case of a VR headset, the other display may display the virtual reality environment in which the user finds themselves or may display a solid color e.g., black) or other effect to obscure the eve.

If the ophthalmic test requires user participation, at operation 410, the messaging application 546 provides a user prompt, The user prompt may be an audible prompt provided via a speaker in the client device 228, the glasses 100, 300, or a connected loudspeaker such as wired or wireless earbuds or a headset. The user prompt may for example instruct the user to “Look up” or “Look down” or “Look at target X” or “Follow target X,” where target X is a stationary or moving target displayed on the display corresponding to the eye under test.

At operation 412, user compliance with the user prompt is determined by the messaging application 546. In one example the user's compliance may be determined by the messaging application 546 recognizing a confirming audible response from the user, received by a microphone in the client device 228, the glasses 100, 300, or a connected microphone for example such as in wired or wireless earbuds or a headset. The user may be prompted to say “done” or “ready” in response to a prompt, In another example, image recognition or object detection and tracking may be performed on the eye, to determine that the eye is positioned correctly for image capture.

In response to receiving an indication of user compliance, illumination by light emitters 318 or optical assembly 316 is provided to the eye(s) under consideration if required and not already provided, and one or more images are captured by one or more of the cameras 314 at operation 414.

At operation 416, the messaging application 546 determines whether or not the health test of the eye under consideration is complete. The test may for example be complete if all required steps have been performed or all required images have been captured. If the test is not complete, the method returns to operation 410 where the next prompt is provided by the messaging application 546.

If the messaging application 546 determines that the health test of the eye under consideration is complete, the method proceeds to operation 418 where the messaging application 546 checks whether only one eye has been tested or whether both eyes have been tested. If only one eye has been tested, the method proceeds at operation 420 where the test procedure is switched to the second eye. Any obscuring or other display content or color shown to the eye that was previously not under test is switched to the eye for which the test has just been completed.

The flowchart 400 then continues as before through operation 410 to operation 416 to test the other eye. After both eyes have been tested as verified at operation 418, the messaging application 546 analyzes the image(s) at operation 422. This may be done on a single image, multiple images, or a composite image of a feature of each eye generated from multiple images of the relevant eye. The images may be analyzed using any known image processing technique for detecting health problems with an eye. The particular image processing technique will differ according to the health problem in question.

The image(s) may also for example may be analyzed using a machine learning classification scheme to identify images indicative of health problems with the eye, The machine learning classification scheme may comprise knowledge of a set of predefined health problems for an eye. The messaging application 546 may then analyze the images using the machine learning classification scheme to identify at least one health problem applicable to the eye based on the images.

Further, the image(s) may not be processed by the messaging application 546 solely to determine the presence of a particular ophthalmic issue selected by the user for screening, but may automatically be analyzed to determine the presence of any of a number of potential ophthalmic issues in addition to the selected ophthalmic issue. Still further, the user interface may not provide specifically-identified ophthalmic issues for user selection, but may instead provide a generic option to scan for eye health issues, in which case the image(s) may be scanned for all available ophthalmic issues that are supported by the messaging application 546. If required for different ophthalmic issues, multiple images or sets of images may automatically be captured with different exposure, illumination or other settings, or user prompts for each ophthalmic issue.

In certain cases it may be possible to obtain suitable images of both eyes at the same time. In such a case, both eyes will be illuminated and imaged simultaneously and it will not be necessary to check at operation 418 whether or not both eyes have been tested.

Additionally, while not strictly an ophthalmic test, an image of a grid projected onto the retina of one or both of the eyes may be processed using known techniques, to determine visual acuity.

After both eyes have been tested, the test results are then output at operation 424. This may be done by displaying the results of the ophthalmic test on the glasses 100, 300, by displaying the results on a display of the client device 228, audibly, or by any other means. In the event that a potential ophthalmic issue has been detected, the results may merely be an instruction or recommendation that the advice of a medical professional be sought. The results may also be compared against previous test results to illustrate any degradation. The results and the underlying or intermediate data (e.g. the captured images) may be transmitted to an eye specialist for review or record-keeping. The user may also be given options to save the test results, post them to a social networking site, or transmit them in a chat session.

The flowchart 400 then ends at operation 426. The messaging application 546 may for example then revert to a default user interface.

FIG. 5 is a block diagram 500 illustrating a software architecture 504, which can be installed on any one or more of the devices described herein. The software architecture 504 is supported by hardware such as a machine 502 that includes processors 520, memory 526, and I/O components 538. In this example, the software architecture 504 can be conceptualized as a stack of layers, where each layer provides a particular functionality. The software architecture 504 includes layers such as an operating system 512, libraries 508, frameworks 510, and applications 506. Operationally, the applications 506 invoke API calls 550 through the software stack and receive messages 552 in response to the API calls 550.

The operating system 512 manages hardware resources and provides common services. The operating system 512 includes, for example, a kernel 514, services 516, and drivers 522 The kernel 514 acts as an abstraction layer between the hardware and the other software layers. For example, the kernel 514 provides memory management, processor management (e.g., scheduling), component management, networking, and security settings, among other functionality. The services 516 can provide other common services for the other software layers. The drivers 522 are responsible for controlling or interfacing with the underlying hardware. For instance, the drivers 522 can include display drivers, camera drivers, BLUETOOTH® or BLUETOOTH® Low Energy drivers, flash memory drivers, serial communication drivers (e.g., Universal Serial Bus (USB) drivers), Wi-Fi® drivers, audio drivers, power management drivers, and so forth.

The libraries 508 provide a low-level common infrastructure used by the applications 506. The libraries 508 can include system libraries 518 (e.g., C standard library) that provide functions such as memory allocation functions, string manipulation functions, mathematic functions, and the like. In addition, the libraries 508 can include API libraries 524 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 508 can also include a wide variety of other libraries 528 to provide many other APIs to the applications 506.

The frameworks 510 provide a high-level common infrastructure that is used by the applications 506. For example, the frameworks 510 provide various graphical user interface (GUI) functions, high-level resource management, and high-level location services. The frameworks 510 can provide a broad spectrum of other APIs that can be used by the applications 506, some of which may be specific to a particular operating system or platform.

In an example, the applications 506 may include a home application 536, a contacts application 530, a browser application 532, a book reader application 534, a location application 542, a media application 544, a messaging application 546, a game application 548, and a broad assortment of other applications such as third-party applications 540. The applications 506 are programs that execute functions defined in the programs. Various programming languages can be employed to create one or more of the applications 506, structured in a variety of manners, such as object-oriented programming languages (e.g., Objective-C, Java, or C++) or procedural programming languages (e.g., C or assembly language). In a specific example, the third-party applications 540 (e.g., applications developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party applications 540 can invoke the API calls 550 provided by the operating system 512 to facilitate functionality described herein.

FIG. 6 is a diagrammatic representation of a machine 600 within which instructions 610 (e.g., software, a program, an application, an applet, an app, or other executable code) for causing the machine 600 to perform any one or more of the methodologies discussed herein may be executed. For example, the instructions 610 may cause the machine 600 to execute any one or more of the methods described herein. The instructions 610 transform the general, non-programmed machine 600 into a particular machine 600 programmed to carry out the described and illustrated functions in the manner described. The machine 600 may operate as a standalone device or may be coupled (e.g., networked) to other machines. In a networked deployment, the machine 600 may operate in the capacity of a server machine or a client machine in a server-client network environment, or as a peer machine in a peer-to-peer (or distributed) network environment. The machine 600 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 610, sequentially or otherwise, that specify actions to be taken by the machine 600. Further, while only a single machine 600 is illustrated, the term “machine” shall also be taken to include a collection of machines that individually or jointly execute the instructions 610 to perform any one or more of the methodologies discussed herein.

The machine 600 may include processors 602, memory 604, and I/O components 606, which may be configured to communicate with each other via a bus 644. In an example, the processors 602 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 608 and a processor 612 that execute the instructions 610. The term “processor” is intended to include multi-core processors that may comprise two or more independent processors (sometimes referred to as “cores”) that may execute instructions contemporaneously. Although FIG. 6 shows multiple processors 602, the machine 600 may include a single processor with a single core, a single processor with multiple cores (e.g., a multi-core processor), multiple processors with a single core, multiple processors with multiples cores, or any combination thereof.

The memory 604 includes a main memory 614, a static memory 616, and a storage unit 618, both accessible to the processors 602 via the bus 644. The main memory 604, the static memory 616, and storage unit 618 store the instructions 610 embodying any one or more of the methodologies or functions described herein. The instructions 610 may also reside, completely or partially, within the main memory 614, within the static memory 616, within machine-readable medium 620 within the storage unit 618, within at least one of the processors 602 (e.g., within the processor's cache memory), or any suitable combination thereof, during execution thereof by the networked system 200.

The I/O components 606 may include a wide variety of components to receive input, provide output, produce output, transmit information, exchange information, capture measurements, and so on. The specific I/O components 606 that are included in a particular machine will depend on the type of machine. For example, portable machines such as mobile phones may include a touch input device or other such input mechanisms, white a headless server machine will likely not include such a touch input device. It will be appreciated that the I/O components 606 may include many other components that are not shown in FIG. 6. In various examples, the I/O components 606 may include output components 628 and input components 632. The output components 628 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 632 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further examples, the I/O components 606 may include biometric components 634, motion components 636, environmental components 638, or position components 640, among a wide array of other components. For example, the biometric components 634 include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 636 include acceleration sensor components accelerometer), gravitation sensor components, rotation sensor components (e.g., gyroscope), and so forth. The environmental components 638 include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 640 include location sensor components (e.g., a OP receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 606 further include communication components 642 operable to couple the networked system 200 to a network 622 or devices 624 via a coupling 630 and a coupling 626, respectively. For example, the communication components 642 may include a network interface component or another suitable device to interface with the network 622. In further examples, the communication components 642 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 624 may be another machine or any of a wide variety of peripheral devices (e.g., a peripheral device coupled via a USB).

Moreover, the communication components 642 may detect identifiers or include components operable to detect identifiers. For example, the communication components 642 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 642, such as location via Internet Protocol (IP) geolocation, location via Wi-Fi® signal triangulation, location via detecting an NFC beacon signal that may indicate a particular location, and so forth.

The various memories (e.g., memory 604, main memory 614, static memory 616, and/or memory of the processors 602) and/or storage unit 618 may store one or more sets of instructions and data structures (e.g., software) embodying or used by any one or more of the methodologies or functions described herein. These instructions (e.g., the instructions 610), when executed by processors 602, cause various operations to implement the disclosed examples.

The instructions 610 may be transmitted or received over the network 622, using a transmission medium, via a network interface device (e.g., a network interface component included in the communication components 642) and using any one of a number of well-known transfer protocols (e.g., hypertext transfer protocol (HTTP)). Similarly, the instructions 610 may be transmitted or received using a transmission medium via the coupling 626 (e.g., a peer-to-peer coupling) to the devices 624.

A “carrier signal” refers to any intangible medium that is capable of storing, encoding, or carrying instructions for execution by the machine, and includes digital or analog communications signals or other intangible media to facilitate communication of such instructions, Instructions may be transmitted or received over a network using a transmission medium via a network interface device.

A “client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network,

A “communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (CPRS) technology, Enhanced. Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

A “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC), A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry e.g., configured by software), may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. in examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

A “computer-readable medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

An “ephemeral message” refers to a message that is accessible for a time-limited duration. An ephemeral message may be a text, an image, a video and the like. The access time for the ephemeral message may be set by the message sender. Alternatively, the access time may be a default setting or a setting specified by the recipient. Regardless of the setting technique, the message is transitory.

A “machine-storage medium” refers to a single or multiple storage devices and/or media (e.g., a centralized or distributed database, and/or associated caches and servers) that store executable instructions, routines and/or data. The term shall accordingly be taken to include, but not be limited to, solid-state memories, and optical and magnetic media, including memory internal or external to processors, Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

A “processor” refers to any circuit or virtual circuit (a physical circuit emulated by logic executing on an actual processor) that manipulates data values according to control signals (e.g., “commands”, “op codes”, “machine code”, and so forth) and which produces corresponding output signals that are applied to operate a machine. A processor may, for example, be a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC) or any combination thereof, A processor may further be a multi-core processor having two or more independent processors (sometimes referred to as “cores”) that may execute instructions contemporaneously.

A “signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal, The terms “transmission medium” and “signal medium” mean the same thing and may be used interchangeably in this disclosure.

Changes and modifications may be made to the disclosed examples without departing from the scope of the present disclosure. These and other changes or modifications are intended to be included within the scope of the present disclosure, as expressed in the following claims.

What is claimed is:

1. A method of conducting an ophthalmic evaluation using a head-worn device including one or more display devices and one or more cameras facing towards an eye of a user in use, comprising:
displaying an ophthalmic evaluation user interface item;
receiving user selection of the user interface item;
illuminating an eye of the user;
capturing at least one image of the eye of the user;
analyzing the at least one image to assess the health of the eye of the user; and
providing an output of the analysis of the at least one image to the user.

2. The method of claim 1, wherein the illuminating is provided by light emitted from the one or more display devices.

3. The method of claim 1, wherein the head-worn device includes light emitters, the illuminating being provided by the light emitters.

4. The method of claim 1, further comprising:
generating audible ophthalmic evaluation prompts on an audio speaker associated with the head-worn device; and
determining compliance with the audible ophthalmic evaluation prompts prior to capturing the at least one image.

5. The method of claim 4, wherein the audible ophthalmic evaluation prompts comprise instructions to look in a certain direction; and
determining compliance is performed by image recognition performed on frames of an image stream received from the one or more cameras.

6. The method of claim 4, further comprising:
displaying a feature on the display device,
wherein the ophthalmic evaluation prompts comprise instructions to look at the feature displayed on the display device.

7. The method of claim 1, wherein determining compliance is performed by receipt of confirmatory user input.

8. The method of claim 1, wherein capturing at least one image of the eye of the user comprises:
capturing multiple images of the eye of the user; and
generating a composite image of the eye of the user from the multiple images.

9. The method of claim 8, further comprising:
generating audible ophthalmic evaluation prompts on an audio speaker associated with the head-worn device; and
determining compliance with each audible ophthalmic evaluation prompt prior to capturing each of the multiple images.

10. The method of claim 8, wherein the ophthalmic evaluation user interface item comprises a plurality of ophthalmic evaluations available for user selection, the method further comprising:
receiving user selection of a specific ophthalmic evaluation; and
setting illumination and camera exposure settings based on the specific ophthalmic evaluation.

11. A computer system including a head-worn device, the computer system comprising:
one or more processors;
one or more display devices;
one or more cameras facing towards an eye of a user in use; and
a memory storing instructions that, when executed by the one or more processors, configure the computer system to perform operations comprising:
displaying an ophthalmic evaluation user interface item on a display device;
receiving user selection of the user interface item;
illuminating an eye of the user;
capturing at least one image of the eye of the user; and
analyzing the at least one image to assess the health of the eye of the user.

12. The computer system of claim 11, wherein the illuminating is provided by light emitted from the one or more display devices.

13. The computer system of claim 11, wherein the instructions further configure the computer system to perform operations comprising:
generating audible ophthalmic evaluation prompts on an audio speaker associated with the head-worn device; and
determining compliance with the audible ophthalmic evaluation prompts prior to capturing the at least one image.

14. The computer system of claim 11, wherein capturing at least one image of the eye of the user comprises:
capturing multiple images of the eye of the user; and
generating a composite image of the eye of the user from the multiple images.

15. The computer system of claim 14, wherein the instructions further configure the computer system to perform operations comprising:
generating audible ophthalmic evaluation prompts on an audio speaker associated with the head-worn device; and
determining compliance with each audible ophthalmic evaluation prompt prior to capturing each of the multiple images.

16. The computer system of claim 14, wherein the ophthalmic evaluation user interface item comprises a plurality of ophthalmic evaluations available for user selection, wherein the instructions further configure the system to perform operations comprising:
receiving user selection of a specific ophthalmic evaluation; and
setting illumination and camera exposure settings based on the specific ophthalmic evaluation.

17. A non-transitory computer-readable storage medium, the computer-readable storage medium including instructions that when executed by a computer system including a head-worn device including one or more display devices and one or more cameras facing towards an eye of a user in use, cause the computer system to perform operations comprising:
displaying an ophthalmic evaluation user interface item on a display device;
receiving user selection of the user interface item;
illuminating an eye of the user;
capturing at least one image of the eye of the user; and
analyzing the at least one image to assess the health of the eye of the user.

18. The computer-readable storage medium of claim 17, wherein the instructions further configure the computer system to perform operations comprising:
generating audible ophthalmic evaluation prompts on an audio speaker associated with the head-worn device; and
determining compliance with the audible ophthalmic evaluation prompts prior to capturing the at least one image.

19. The computer-readable storage medium of claim 17, wherein capturing at least one image of the eye of the user comprises:
capturing multiple images of the eye of the user; and
generating a composite image of the eye of the user from the multiple images.

20. The computer-readable storage medium of claim 19, wherein the instructions further configure the computer system to perform operations comprising:
generating audible ophthalmic evaluation prompts on an audio speaker associated with the head-worn device; and
determining compliance with each audible ophthalmic evaluation prompt prior to capturing each of the multiple images.
